SEQUENCE LISTING (8 sequences)

SEQ ID NO: 1 - DNA, 1846 bp, Amaranthus hybridus
tcatcatcttcttctcaatcacctaaacctaaacctccttccgctactataactcaatcaccttcgtctctcaccgatgataaaccctcttcttttgtttcccgatttagccctgaagaacccagaaaaggttgcgatgttctcgttgaagctcttgaacgtgaaggtgttaccgatgtttttgcttaccctggtggagcatccatggaaattcatcaagctcttactcgttctaatatcattagaaatgttcttcctcgacatgaacaaggtggggttttcgctgctgaaggctacgctcgtgctactggacgcgttggagtttgtattgccacttctggtccaggtgctactaatcttgtttctggtcttgctgatgcacttcttgactcagtccctcttgtcgccattactgggcaagttccccggcgtatgattggtactgatgcttttcaagagactccaattgttgaggtaactcgatccattaccaagcataattatttggtgttagatgttgaggatattcctagaattgttaaggaagctttctttttagctaattctggtagacctggacctgttttgattgatattcctaaagatattcagcaacaattagttgttcctaattgggaacagcccattaaattgggtgggtatctttctaggttgcctaaacccacttattctgctaatgaagagggacttcttgatcaaattgtaaggttagtgggtgagtctaagagacctgtgctgtatactggaggtgggtgtttgaattctagtgaagaattgaggaaatttgtcgaattgacaggtattccggtggctagtactttaatggggttgggggctttcccttgtactgatgatttatctcttcatatgttgggaatgcacgggactgtgtacgcgaattacgcggttgataaggccgatttgttgcttgcttttggggttaggtttgatgaacgagtgactggtaagctcgaggcgtttgctagccgggctaagattgtgcacatcgatatcgattctgctgaaatcgggaagaataagcaacctcatgtgtcgatttgtggtgatgttaaagtggcattacaggggttgaataagattttggaatctagaaaaggaaaggtgaaattggatttctctaattggagggaggagttgaatgagcagaaaaagaagtttcctttgagttttaagactttcggggatgcaattcctccgcaatacgccattcaggttcttgacgagttgacgaagggcgatgcggttgtaagtactggtgttgggcagcaccaaatgtgggctgcccaattctataagtaccgaaatcctcgccaatggctgacctcgggtggtttgggggctatggggtttggtctaccagctgctattggagctgctgttgctcgaccagatgcggtggttgtagacattgatggggatgggagttttatcatgaatgttcaagagttggctacgattagggtagagaatctcccggttaaaatcatgctcttgaacaatcaacatttaggtatggttgttcaatgggaagatcgattttacaaagctaaccgggcacatacatacctcgggaatccttccaattcttccgaaatcttcccggatatgctcaaatttgctgaagcatgtgatataccagcagcccgtgttaccaaggtgagcgatttaagggctgcaattcaaacaatgttggatactccaggaccgtatctgctggatgtaatcgtaccacatcaggagcatgtgctgcctatgatccctagcggtgccgccttcaaggacaccataacagagggtgatggaa

SEQ ID NO: 2 - protein, 614 aa, Amaranthus hybridus
SSSSQSPKPKPPSATITQSPSSLTDDKPSSFVSRFSPEEPRKGCDVLVEALEREGVTDVFAYPGGASMEIHQALTRSNIIRNVLPRHEQGGVFAAEGYARATGRVGVCIATSGPGATNLVSGLADALLDSVPLVAITGQVPRRMIGTDAFQETPIVEVTRSITKHNYLVLDVEDIPRIVKEAFFLANSGRPGPVLIDIPKDIQQQLVVPNWEQPIKLGGYLSRLPKPTYSANEEGLLDQIVRLVGESKRPVLYTGGGCLNSSEELRKFVELTGIPVASTLMGLGAFPCTDDLSLHMLGMHGTVYANYAVDKADLLLAFGVRFDERVTGKLEAFASRAKIVHIDIDSAEIGKNKQPHVSICGDVKVALQGLNKILESRKGKVKLDFSNWREELNEQKKKFPLSFKTFGDAIPPQYAIQVLDELTKGDAVVSTGVGQHQMWAAQFYKYRNPRQWLTSGGLGAMGFGLPAAIGAAVARPDAVVVDIDGDGSFIMNVQELATIRVENLPVKIMLLNNQHLGMVVQWEDRFYKANRAHTYLGNPSNSSEIFPDMLKFAEACDIPAARVTKVSDLRAAIQTMLDTPGPYLLDVIVPHQEHVLPMIPSGAAFKDTITEGDG

SEQ ID NO: 3 - DNA, 1930 bp, Amaranthus hybridus
tcatcatcttcttctcaatcacctaaacctaaacctccttccgctactataactcaatcaccttcgtctctcaccgatgataaaccctcttcttttgtttcccgatttagccctgaagaacccagaaaaggttgcgatgttctcgttgaagctcttgaacgtgaaggtgttaccgatgtttttgcttaccctggtggagcatccatggaaattcatcaagctcttactcgttctaatatcattagaaatgttcttcctcgacatgaacaaggtggggttttcgctgctgaaggctacgctcgtgctactggacgcgttggagtttgtattgccacttctggtccaggtgctactaatcttgtttctggtcttgctgatgcacttcttgactcagtccctcttgtcgccattactgggcaagttccccggcgtatgattggtactgatgcttttcaagagactccaattgttgaggtaactcgatccattaccaagcataattatttggtgttagatgttgaggatattcctagaattgttaaggaagctttctttttagctaattctggtagacctggacctgttttgattgatattcctaaagatattcagcaacaattagttgttcctaattgggaacagcccattaaattgggtgggtatctttctaggttgcctaaacccacttattctgctaatgaagagggacttcttgatcaaattgtaaggttagtgggtgagtctaagagacctgtgctgtatactggaggtgggtgtttgaattctagtgaagaattgaggaaatttgtcgaattgacaggtattccggtggctagtactttaatggggttgggggctttcccttgtactgatgatttatctcttcatatgttgggaatgcacgggactgtgtacgcgaattacgcggttgataaggccgatttgttgcttgcttttggggttaggtttgatgatcgagtgactggtaagctcgaggcgtttgctagccgggctaagattgtgcacatcgatatcgattctgctgaaatcgggaagaataagcaacctcatgtgtcgatttgtggtgatgttaaagtggcattacaggggttgaataagattttggaatctagaaaaggaaaggtgaaattggatttctctaattggagggaggagttgaatgagcagaaaaagaagtttcctttgagttttaagactttcggggatgcaattcctccgcaatacgccattcaggttcttgacgagttgacgaagggcgatgcggttgtaagtactggtgttgggcagcaccaaatgtgggctgcccaattctataagtaccgaaatcctcgccaatggctgacctcgggtggtttgggggctatggggtttggtctaccagctgctattggagctgctgttgctcgaccagatgcggtggttgtagacattgatggggatgggagttttatcatgaatgttcaagagttggctacgattagggtagagaatctcccggttaaaatcatgctcttgaacaatcaacatttaggtatggttgttcaatgggaagatcgattttacaaagctaaccgggcacatacatacctcgggaatccttccaattcttccgaaatcttcccggatatgctcaaatttgctgaagcatgtgatataccagcagcccgtgttaccaaggtgagcgatttaagggctgcaattcaaacaatgttggatactccaggaccgtatctgctggatgtaatcgtaccacatcaggagcatgtgctgcctatgatccctagcggtgccgccttcaaggacaccataacagagggtgatggaagaagggcttattagttggttggagatctttatagaggagaagcttttttgtatgtatgttagtagttccataaacttctatatt

SEQ ID NO: 4 - protein, 619 aa, Amaranthus hybridus
SSSSSQSPKPKPPSATITQSPSSLTDDKPSSFVSRFSPEEPRKGCDVLVEALEREGVTDVFAYPGGASMEIHQALTRSNIIRNVLPRHEQGGVFAAEGYARATGRVGVCIATSGPGATNLVSGLADALDDSVPLVAITGQVPRRMIGTDAFQETPIVEVTRSITKHNYLVLDVEDIPRIVKEAFFLANSGRPGPVLIDIPKDIQQQLVVPNWEQPIKLGGYLSRLPKPTYSANEEGLLDQIVRLVGESKRPVLYTGGGCLNSSEELRKFVELTGIPVASTLMGLGAFPCTDDLSLHMLGMHGTVYANYAVDKADLLLAFGVRFDDRVTGKLEAFASRAKIVHIDIDSAEIGKNKQPHVSICGDVKVALQGLNKILESRKGKVKLDFSNWREELNEQKKKFPLSFKTFGDAIPPQYAIQVLDELTKGDAVVSTGVGQHQMWAAQFYKYRNPRQWLTSGGLGAMGFGLPAAIGAAVARPDAVVVDIDGDGSFIMNVQELATIRVENLPVKIMLLNNQHLGMVVQWEDRFYKANRAHTYLGNPSNSSEIFPDMLKFAEACDIPAARVTKVSDLRAAIQTMLDTPGPYLLDVIVPHQEHVLPMIPSGAAFKDTITEGDGRRAY

SEQ ID NO: 5 - protein, 11 aa, Amaranthus hybridus
GVRFDERVTGK

SEQ ID NO: 6 - protein, 11 aa, Amaranthus hybridus
GVRFDDRVTGK

SEQ ID NO: 7 - protein, 618 aa, Amaranthus hybridus
SSSSQSPKPKPPSATITQSPSSLTDDKPSSFVSRFSPEEPRKGCDVLVEALEREGVTDVFAYPGGASMEIHQALTRSNIIRNVLPRHEQGGVFAAEGYARATGRVGVCIATSGPGATNLVSGLADALLDSVPLVAITGQVPRRMIGTDAFQETPIVEVTRSITKHNYLVLDVEDIPRIVKEAFFLANSGRPGPVLIDIPKDIQQQLVVPNWEQPIKLGGYLSRLPKPTYSANEEGLLDQIVRLVGESKRPVLYTGGGCLNSSEELRKFVELTGIPVASTLMGLGAFPCTDDLSLHMLGMHGTVYANYAVDKADLLLAFGVRFDERVTGKLEAFASRAKIVHIDIDSAEIGKNKQPHVSICGDVKVALQGLNKILESRKGKVKLDFSNWREELNEQKKKFPLSFKTFGDAIPPQYAIQVLDELTKGDAVVSTGVGQHQMWAAQFYKYRNPRQWLTSGGLGAMGFGLPAAIGAAVARPDAVVVDIDGDGSFIMNVQELATIRVENLPVKIMLLNNQHLGMVVQWEDRFYKANRAHTYLGNPSNSSEIFPDMLKFAEACDIPAARVTKVSDLRAAIQTMLDTPGPYLLDVIVPHQEHVLPMIPSGAAFKDTITEGDGRRAY

SEQ ID NO: 8 - protein, 618 aa, Amaranthus hybridus
SSSSQSPKPKPPSATITQSPSSLTDDKPSSFVSRFSPEEPRKGCDVLVEALEREGVTDVFAYPGGASMEIHQALTRSNIIRNVLPRHEQGGVFAAEGYARATGRVGVCIATSGPGATNLVSGLADALDDSVPLVAITGQVPRRMIGTDAFQETPIVEVTRSITKHNYLVLDVEDIPRIVKEAFFLANSGRPGPVLIDIPKDIQQQLVVPNWEQPIKLGGYLSRLPKPTYSANEEGLLDQIVRLVGESKRPVLYTGGGCLNSSEELRKFVELTGIPVASTLMGLGAFPCTDDLSLHMLGMHGTVYANYAVDKADLLLAFGVRFDDRVTGKLEAFASRAKIVHIDIDSAEIGKNKQPHVSICGDVKVALQGLNKILESRKGKVKLDFSNWREELNEQKKKFPLSFKTFGDAIPPQYAIQVLDELTKGDAVVSTGVGQHQMWAAQFYKYRNPRQWLTSGGLGAMGFGLPAAIGAAVARPDAVVVDIDGDGSFIMNVQELATIRVENLPVKIMLLNNQHLGMVVQWEDRFYKANRAHTYLGNPSNSSEIFPDMLKFAEACDIPAARVTKVSDLRAAIQTMLDTPGPYLLDVIVPHQEHVLPMIPSGAAFKDTITEGDGRRAY

(19) United States
(12) Patent Application Publication
(10) Pub. No.: US 2006/0130172 A1
(43) Pub. Date: Jun. 15, 2006

(54) Gene encoding resistance to acetolactate synthase-inhibiting herbicides

(76) Inventors: Cory Whaley, Seaford, DE (US); Henry P. Wilson, Belle Haven, VA (US); James H. Westwood, Blacksburg, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/540,971

(22) PCT Filed: Jan. 9, 2004
(86) PCT No.: PCT/US04/00363

§ 371 Date: Dec. 19, 2005

Related U.S. Application Data
(60) Provisional application No. 60/438,801, filed on Jan. 9, 2003.

(51) Int. Cl. A01H 1/00; C12N 9/10; C12N 15/82; C12N 15/87

(52) U.S. Cl. 800/278; 435/193; 435/468

(57) ABSTRACT

A mutant acetolactate synthase (ALS) enzyme that confers cross-resistance to all sulfonylurea, imidazolinone, pyrimidinyloxybenzoate, triazolopyrimidine and sulfonylamino-carbonyl-triazolinone herbicides is provided. The mutant enzyme contains an aspartic acid to glutamic acid substitution mutation at a newly identified conserved region of the ALS enzyme. A gene encoding the enzyme is also provided, as are transgenic plants that have been genetically engineered to contain and express the gene. The transgenic plants are cross-resistant to sulfonylurea, imidazolinone, pyrimidinyloxybenzoate, triazolopyrimidine and sulfonylamino-carbonyl-triazolinone herbicides.

### BACKGROUND OF THE INVENTION

1. Field of the Invention

The invention generally relates to herbicide resistance. In particular, the invention provides a mutant acetolactate synthase (ALS) gene that confers cross-resistance to all sulfonylurea, imidazolinone, pyrimidinyloxybenzoate, triazolopyrimidine, and sulfonylamino-carbonyl-triazolinone herbicides.

2. Background of the Invention

Herbicides have simplified weed management in agriculture and provide a highly effective means of keeping weed populations at acceptable levels. However, crop sensitivity to numerous herbicides limits the use of these herbicides to tolerant crops only. Certain herbicides currently registered for use in crops still result in injury even at normal use rates. Crop injury increases when higher application rates are required to manage large weeds or heavy infestations that are beyond control with normal use rates. In extreme situations, the only effective herbicides available may result in significant crop injury. Furthermore, residual herbicides remaining in the soil are often a problem with rotation to a sensitive crop the following season, which may hinder the use of effective herbicides based on rotational restrictions. Modification of crop plants to create herbicide resistance has been an effective tool to increase weed control, minimize crop injury, allow applications of herbicides in crops with previous sensitivity, reduce herbicide inputs, and make use of more environmentally sound herbicide options. Transgenic crops resistant to a specific herbicide have been developed by transformation with target enzymes that are insensitive to a specific herbicide.

Acetolactate synthase (ALS) is an enzyme that catalyzes the initial step in the branched chain amino acid biosynthetic pathway. ALS is the target site of several classes of unrelated herbicide chemistries, including sulfonylureas (SU), imidazolinones (IMI), pyrimidynyloxybenzoates (POB), triazolopyrimidines (TP), and sulfonylamino-carbonyl-triazolinones (Table 1). Currently, ALS-inhibiting herbicides comprise the largest mode-of-action group in use due to broad-spectrum weed control in a variety of crops at very low application rates. In addition, ALS-inhibiting herbicides have very low mammalian toxicity. These characteristics have increased the importance of these herbicides in production agriculture and have attracted the development of ALS-resistant crops.

A single nucleotide mutation in the ALS enzyme is capable of conferring herbicide specific resistance. Mutations have been identified in five highly conserved domains along the DNA sequence coding for the ALS enzyme in higher plants. Each domain contains a single variable residue, that when substituted, confers resistance to specific ALS-inhibiting herbicides. In most cases, a single substitution results in target-site cross-resistance differences between ALS-inhibiting herbicide chemistries (Table 2). A substitution reported at Ala₁₃₃ in domain C of common cocklebur resulted in resistance to IMI herbicides only. The identical mutation was found in a commercial field corn hybrid, ICI 8532 IT, and sugar beet line Sur, which are crops resistant to only IMI herbicides (Bernasconi et al., J. Biol. Chem. (1995) 270:17381-17385; Wright et al., Weed Sci. (1998) 46:13-23). Substitutions at Pro₁₉₇ in domain A have resulted in a high level of resistance to SU herbicides with little or no resistance to IMI herbicides (Guttieri et al., Weed Sci. (1992) 40:670-676; Guttieri et al., Weed Sci (1995) 43:175-178; Boutsalis et al., Pestic. Sci. (1999) 55:507-516). A domain E mutation of Ser₆₇₀ to Asp resulted in a high level of resistance to IMI herbicides with low SU resistance (Devine and Eberlein, Herbicide Activity: Toxicology, Biochemistry and Molecular Biology (1997) 159-185).

High-level cross-resistance between ALS-herbicide chemistries has been shown previously with field isolated common cocklebur (Xanthium strumarium) biotypes exposed to several years of ALS selection pressure (Bemasconi et al., J. Biol. Chem. (1995) 270:17381-17). The isolated protein from one resistant biotype had a Trp₅₅₂ to Leu mutation as compared to the susceptible population. This mutation corresponded to the Trp₅₄₂ to Leu mutation in a commercial corn hybrid, Pioneer 3180 IR, which exhibited broad-range tolerance to ALS-inhibiting herbicides. A second common cocklebur field isolate had a substitution of Ala₁₈₃ to Val in Domain D that conferred similar cross-resistance patterns to the mutation found in domain B (Woodworth et al., Plant Physiol. (1996) 111:415).

TABLE 1
Representative examples of sulfonylurea, imidazolinone,
pyrimidinyloxybenzoate, and triazolopyrimidine ALS-inhibiting
herbicides and corresponding chemical names.

| ALS-Inhibitor | | |
|---|---|---|
| Family | Common Name | Chemical Name |
| Sulfonylurea | chlorimuron | 2-[[[[(4-chloro-6- |
| | | methoxy-2-pyrimidinyl)- |
| | | amino]carbonyl]amino]- |
| | | sulfonyl]benzoic acid |
| | thifensulfuron | 3-[[[[(4-methoxy- |
| | | 6-methyl-1,3,5-triazin-2- |
| | | yl)amino]carbonyl]- |
| | | amino]sulfonyl]- |
| | | 2-thiophenecarboxylic acid |
| | trifloxysulfuron | N-[(4,6-dimehoxy-2- |
| | | pyrimidinyl)carbamoyl]- |
| | | 3-(2,2,2-trifluoroethoxy)- |
| | | pyridin-2-sulfonamide |
| | nicosulfuron | 2-[[[[(4,6- |
| | | dimethoxy-2-pyrimidinyl)- |
| | | amino]carbonyl]- |
| | | amino]sulfonyl]-N,N- |
| | | dimethyl-3-pyridine- |
| | | carboxamide |
| Imidazolinone | imazethapyr | 2-[4,5-dihydro-4-methyl- |
| | | 4-(1-methylethyl)-5-oxo-1H- |
| | | imidazol-2-yl]-5-ethyl- |
| | | 3-pyridinecarboxylic acid |
| | imazaquin | 2-[4,5-dihydro-4-methyl- |
| | | 4-(1-methylethyl)-5-oxo- |
| | | 1H-imidazol-2-yl]-3- |
| | | quinolinecarboxylic acid |
| | imazapyr | (±)-2-[4,5-dihydro- |
| | | 4-methyl-4-(1-methylethyl)- |
| | | 5-oxo-1H-imidazol-2-yl]- |
| | | 3-pyridinecarboxylic acid |
| Pyrimidinyloxy- | pyrithiobac | 2-chloro-6-[(4,6-dimethoxy- |
| benzoate | | 2-pyrimidinyl)thio]benzoic |
| | | acid |
| Triazol- | cloransulam | 3-chloro-2-[[(5-ethoxy- |
| pyrimidine | | 7-fluoro[1,2,4]triazolo[- |
| | | 1,5-c]pyrimidin-2yl)- |
| | | sulfonyl]amino]benzoic |
| | | acid |
| | flumetsulam | N-(2,6-difluorophenyl)-5- |
| | | methyl[1,2,4]triazolo- |
| | | [1,5-a]pyrimidine-2- |
| | | sulfonamide |
| Sulfonylamino- | flucarbazone | 4,5-dihydro-3-methoxy-4- |
| carbonyl- | | methyl-5-oxo-N-[[2- |
| triazolinones | | (trifluoromethoxy)phenyl]- |
| | | sulfonyl]-1H-1,2,4- |
| | | triazole-1-carboxamide |
| | propoxycarbazone | methyl 2-[[[(4,5- |
| | | dihydro-4-methyl-5-oxo-3- |
| | | propoxy-1H-1,2,4-triazol- |
| | | 1-yl)carbonyl]amino]- |
| | | sulfonyl]benzoate |

TABLE 2

Common ALS mutations and corresponding levels of resistance conferred to
SU, IMI, and TP herbicides (Devine and Shukla, Crop Prot. (2000)
19:881-889).

Resistance Level

| | | | | | | |
|---|---|---|---|---|---|---|
| Mutation | Domain | Domain Sequence | SU | IMI | TP | Reference |

| | | | | | | |
|---|---|---|---|---|---|---|
| Ala₁₂₂ to Thr | C | VFAYPGGASMEIHQALTRS | Low | High | Low | Bernasconi et al. (1995) |
| | | (SEQ ID NO: 8) | zero | | zero | |
| Pro₁₉₇ to Ala | A | AITGQVPRRMIGT | High | Zero | Mod | Boutsalis et al. (1999) |
| | | (SEQ ID NO: 9) | | | low | |
| Pro₁₉₇ to Thr | | | High | Low | — | Guttieri et al. (1995) |
| | | | | zero | | |
| Pro₁₉₇ to His | | | High | Mod | Low | Guttieri et al. (1992) |
| Pro₁₉₇ to Leu | | | High | Mod | High | Guttieri et al. (1995) |
| | | | | low | | |
| Pro₁₉₇ to Arg | | | High | — | — | Guttieri et al. (1995) |
| Pro₁₉₇ to Ile | | | High | Mod | Mod | Boutsalis et al. (1999) |
| | | | | low | low | |
| Pro₁₉₇ to Gln | | | High | — | — | Guttieri et al. (1995) |
| Pro₁₉₇ to Ser | | | High | Zero | High | Guttieri et al. (1995) |
| Ala₂₀₅ to Asp | D | AFQETP | High | — | — | Hartnett et al. (1990) |
| | | (SEQ ID NO: 10) | | | | Woodworth et al. (1996)(2) |
| Trp₅₉₁ to Leu | B | QWED | High | High | High | Boutsalis et al. (1999) |
| | | (SEQ ID NO: 11) | | | | |
| Ser₆₇₀ to Asp | E | IPSGG | Low | High | Zero | Devine and Eberlein (1997) |
| | | (SEQ ID NO: 12) | | | | |

ALS-resistant crops currently marketed provide herbicide resistance to only a single class of ALS-inhibiting herbicides, either imidazolinone or sulfonylurea classes. There is an ongoing need to develop herbicide resistant crops, and it would be particularly desirable to develop crops with resistance to more than one herbicide. The prior art has thus far failed to meet this need.

### SUMMARY OF THE INVENTION

It is an object of this invention to provide a functional, mutant ALS enzyme that is broadly resistant to ALS-inhibiting herbicide chemistries. Transgenic plants that have been genetically engineered to contain and express a gene encoding the enzyme are thus able to grow and reproduce even after the application of two or more herbicides (even those representing different herbicide families) to which the mutant ALS confers resistance. In contrast, other plants (e.g. weeds) that may be resistant to one family of the herbicides, but are not resistant to other families of ALS-inhibiting herbicides will be inhibited in their growth and reproduction after the application of two or more herbicides. In the mutant enzyme, ALS resistance is conferred by a single amino acid mutation in a conserved region previously unreported along the ALS gene in higher plants. The ALS enzyme of the present invention is cross-resistant to at least four classes of structurally unrelated ALS-inhibiting herbicide chemistries, including imidazolinones, sulfonylureas, pyrimidinyloxybenzoates, triazolopyrimidines, and sulfonylamino-carbonyl-triazolinones. Together, these classes comprise the largest mode-of-action herbicide group, representing over 50 commercial herbicides used in all major crops (eg. corn, wheat, soybean, rice, cotton, and canola) and a wide range of minor crops. This mutation creates an ALS enzyme with resistance to any ALS enzyme-inhibiting herbicide and offers the possibility of creating herbicide-resistant crops with cross-resistance to all herbicides in these groups.

The present invention thus provides a substantially purified acetolactate synthase (ALS) enzyme that confers, in a plant, cross-resistance to multiple herbicides. In one embodiment of the invention, the sequence of the ALS enzyme is SEQ ID NO: 1, or a fragment thereof with ALS activity. At least two of the multiple herbicides may be sulfonylurea, imidazolinone, pyrimidinyloxybenzoate, triazolopyrimidine or sulfonylamino-carbonyl-triazolinone herbicides.

The invention also provides a substantially purified ALS gene encoding an ALS enzyme that confers, in a plant, cross-resistance to multiple herbicides. In one embodiment, the gene is SEQ ID NO: 2 or a fragment thereof encoding a polypeptide with ALS activity. At least two of the multiple herbicides may be sulfonylurea, imidazolinone, pyrimidinyloxybenzoate, triazolopyrimidine or sulfonylamino-carbonyl-triazolinone herbicides.

The invention also provides a method of conferring cross-resistance to multiple herbicides to a plant. The method includes the step of introducing into the plant an expressible gene encoding an ALS enzyme that exhibits cross-resistance to multiple herbicides. The step of introducing the gene into the plant confers cross-resistance to multiple herbicides to the plant. The gene may be SEQ ID NO: 1, or a fragment thereof that encodes a polypeptide having ALS activity.

The invention also provides a transgenic plant that is cross-resistant to multiple herbicides. The transgenic plant is comprised of a host plant that contains an expressible gene that is not naturally present in the plant, and the gene encodes an ALS enzyme that confers cross-resistance to multiple herbicides. The gene may be SEQ ID NO:2, or a fragment thereof that encodes a polypeptide having ALS activity. The multiple herbicides may be sulfonylurea, imidazolinone, pyrimidinyloxybenzoate, triazolopyrimidine or sulfonylamino-carbonyl-triazolinone herbicides. The transgenic plant may be, for example, Arabidopsis, corn, cotton, soybean, rice, wheat, or a forage crop. The ALS enzyme may have an aspartic acid to glutamic acid substitution at position six of a conserved sequence GVRFDDRVTGK (SEQ ID NO: 6).

### BRIEF DESCRIPTION OF THE DRAWINGS

FIG. 1A and B. R11-AMACH sequences. A) nucleotide sequence; B) primary amino acid sequence.

FIG. 2A and B. S-AMACH sequences. A) nucleotide sequence; B) primary amino acid sequence.

FIG. 3. Amino acid sequence alignment of R11-AMACH and S-AMACH ALS gene. The mutation (D to E) is indicated on top of the alignment (#) at position 375 within the highlighted region.

### DETAILED DESCRIPTION OF THE PREFERRED EMBODIMENTS OF THE INVENTION

The present invention provides a mutant acetolactate (ALS) enzyme that confers, when produced by a plant, cross-resistance to multiple herbicides. The enzyme contains a single amino acid substitution ( aspartic acid to glutamic acid, in a newly identified conserved region of the ALS enzyme. The conserved region has the primary amino acid sequence GVRFDDRVTGK, (SEQ ID NO: 6) and the D to E substitution occurs at aspartic acid at position 6, resulting in a mutant conserved sequence GVRFDERVTGK, (SEQ ID NO: 7). In smooth pigweed (Amaranthus hybridus L.) ALS, the D to E substitution is at residue 375; in Arabidopsis, the D to E substitution is at residue 376. Those of skill in the art will recognize that the precise position of the substitution in a full length ALS enzyme may vary from species to species, or from variety to variety due to genetic variation.

However, the mutation is located at amino acid position 6 of the conserved sequence GVRFDDRVTGK, (SEQ ID NO: 6). The “conserved sequence” itself may vary slightly depending on the source, and in particular may have conservative amino acid substitutions, but will generally be in the range of about 90 to 100% homologous to (SEQ ID NO: 6).

Surprisingly, the mutant ALS enzyme, when produced in a plant, renders the plant cross-resistant to multiple classes of herbicides, including imidazolinones, sulfonylureas, pyrimidinyloxybenzoates, triazolopyrimidines and sulfonylamino-carbonyl-triazolinones. The invention also provides a mutant gene that encodes the cross-resistant ALS enzyme, as well as transgenic plants that have been genetically engineered to contain the mutant gene and which thus produce a functional mutant enzyme, and a method for transforming plants with the mutant gene. Such transgenic plants display cross-resistance to multiple herbicides and are able to grow and reproduce even after the application of two or more of the herbicides to which they are cross-resistant.

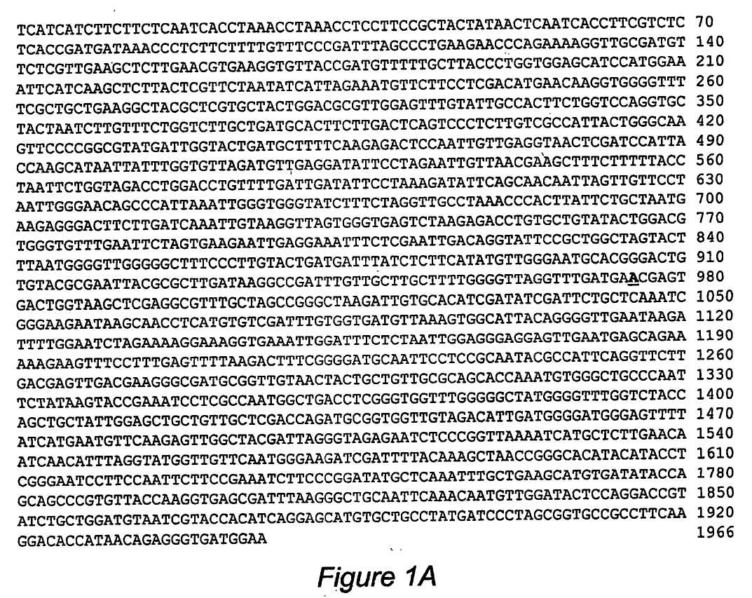

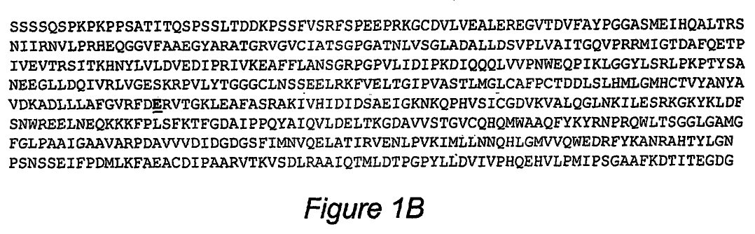

In a preferred embodiment of the invention, the nucleotide sequence that encodes the cross-resistant ALS enzyme is that which is shown in FIG. 1A (SEQ ID NO: 1). The corresponding amino acid sequence is given in FIG. 1B (SEQ ID NO: 2). However, those of skill in the art will recognize that various permutations of the nucleic acid sequence of SEQ ID NO: 1 may also be used to encode an enzyme of the present invention. These include but are not limited to: shorter portions of the DNA molecule which encode truncated ALS enzymes that still possess ALS activity and exhibit cross-resistance to multiple herbicides; nucleic acid sequences that contain various substitutions that, due to the redundancy of the genetic code, still encode an enzyme identical to SEQ ID NO: 2; nucleic acid sequences that are substantially as in SEQ ID NO: 2 but which have been altered for any reason, such as to introduce a convenient restriction enzyme cleavage site, to alter the tertiary structure of the DNA molecule, etc.; various nucleic acid sequences that are substantially homologous to SEQ ID NO: 1 (e.g. that display from about 70% to about 100% homology, and preferably about 80% to 100% homology, and most preferably about 90% to 100% homology) to SEQ ID NO: 1, but still encoding an enzyme with ALS activity and which displays multiple herbicide cross-resistance as described herein. All such DNA molecules, as well as any vectors which include the DNA molecules, are intended to be encompassed within the scope of the present invention.

The invention further encompasses RNA molecules which encode a mutant ALS enzyme of the present invention, for example, mRNA molecules transcribed from a gene encoding an ALS enzyme of the present invention.

Accordingly, the invention also contemplates mutant ALS enzymes that display cross-resistance to multiple herbicides and which have a primary amino acid sequence as in SEQ ID NO: 2. Alternatively, various related but non-identical polypeptide sequences are also contemplated by the present invention, e.g. polypeptides which possess about 70 to 100% homology, or preferably 80 to 100% homology, or most preferably 90 to 100% homology to SEQ ID NO: 2, so long as the related polypeptide displays ALS activity, and exhibits cross-resistance to multiple herbicides. Changes to the sequence may be made for any reason, and may involve conservative or nonconservative amino acid substitutions, or amino acid additions or deletions. For example, residues may be changed by well-known genetic engineering techniques in order to introduce or eliminate sequences susceptible to cleavage by proteases, to non-enzymatic deamidation reactions, to various post-translational modification reactions (e.g. glycosylation, acetylation, etc.), to enhance solubility of the polypeptide, etc. Such mutant ALS enzymes may be approximately a fall length polypeptide such as that of SEQ ID NO: 2. Alternatively, the mutant ALS enzyme may be a truncated version or fragment of the polypeptide which retains ALS activity and multiple herbicide resistance (e.g. a shorter polypeptide that is based on the primary sequence of the fall length ALS gene but has, for example, a portion of the carboxy or amino terminal residues deleted, or which has a portion of intervening sequences between the carboxy and amino termini deleted. Such a fragment would in general possess about 70 to 100% homology to the region of full length ALS to which it corresponds (i.e. the primary sequence of ALS that was not deleted), or preferably about 80 to 100% homology, and most preferably about 90 to 100% homology. Further, the sequence of the mutant ALS enzyme may be genetically engineered to contain other useful sequences, e.g. sequences which serve to target the polypeptide to a location within the cell or within the plant, sequences which facilitate isolation of the protein (e.g. an amino acid tag), and the like. In addition, chimeric fusion proteins in which the mutant ALS enzyme is translated in tandem with or otherwise joined to a related or non-related protein, examples of which include but are not limited to proteins or polypeptides which facilitate tracking of the ALS enzyme (e.g. green fluorescent protein, etc.) or proteins which confer some other useful property to the enzyme or plant, such as antibiotic resistance, or markers such as β-glucoronidaase (GUS), luciferase, β-galactosidase, chloramphenicol acetyl transferase (CAT), octopine, nopaline synthase, NPT-II, etc. All such variations of the mutant ALS enzyme depicted in SEQ ID NO: 2 are intended to be encompassed by the present invention, so long as the variant enzymes display ALS activity and cross-resistance to multiple herbicides.

By “displays ALS activity” we mean that the mutant, genetically engineered enzyme exhibits at least about 50% or more of the level of activity of the corresponding wild type enzyme, when tested under standard conditions for testing ALS activity. Typically, the measurement of ALS activity utilizes a discontinuous colorimetric assay as described by Singh et al., (1988). The assay involves combining enzyme, pyruvate, cofactors, and other additives, followed by a fixed time incubation. The reaction is terminated by addition of sulfuric acid and heated to convert acetolactate to acetoin. The acetoin is converted to a colored complex upon addition of creatine and α-naphthol, as described by Westerfield, (1945). The absorbance of the reaction mixture is measured at 525 nm.

By “herbicide resistance” we mean an inherited ability of a plant to survive and reproduce following treatment with a dose of herbicide that would be lethal to the wild type. This definition includes plants rendered resistant through genetic engineering. By “cross-resistance” or “cross-resistance to multiple herbicides” we mean herbicide resistance to two or more herbicides that have the same general mechanism of action, for example, the inhibition of an enzyme such as ALS. This is in contrast to “multiple herbicide resistance” which is understood to mean herbicide resistance to two or more herbicides that have completely different mechanisms of action.

Herbicides of interest for the present application include but are not limited to sulfonylureas, imidazolinones, pyrimidinyloxybenzoates, triazolopyrimidines, and sulfonylamino-carbonyl-triazolinones, as well as any other classes of herbicides that act through inhibition of the ALS enzyme.

The ALS mutant enzyme of the present invention and the nucleic acid encoding the ALS mutant enzyme of the present invention may be provided in a substantially purified form. By “substantially purified” we mean that the molecule is substantially free of other contaminating matter (such as molecules of other protein, nucleic acids, plant tissue, etc.) which might be associated with the enzyme or nucleic acid of the present invention prior to purification. Those of skill in the art will recognize the standards typically used for assessing purification of an enzyme or nucleic acid, and the means for carrying out such an assessment (e.g. analysis via chromatography, gels, mass spectroscopy, nuclear magnetic resonance, measurement of activity, etc.). The level of purification will generally be greater than about 60%, and preferably greater than about 75%, and most preferably from about 90 to 100% pure, based on, for example, a weight to weight basis of enzyme or nucleic acid to enzyme or nucleic acid plus contaminant(s). The present invention also provides transgenic plants that have been genetically altered (i.e. genetically engineered) to contain and express a gene encoding a mutant ALS enzyme that confers to the plants cross-resistance to multiple herbicides. The expressible gene is not naturally present in the plant, and it is typically introduced into the plant by any of many well-known genetic engineering techniques. The invention further provides a method of conferring herbicide cross-resistance to a plant by introducing into the plant a gene encoding a mutant ALS enzyme of the present invention. The methodology for creating transgenic plants is well developed and well known to those of skill in the art. For example, dicotyledon plants such as soybean, squash, tobacco (Lin et al. 1995), and tomatoes can be transformed by Agrobacterium-mediated bacterial conjugation. (Miesfeld, 1999, and references therein). In this method, special laboratory strains of the soil bacterium Agrobacterium are used as a means to transfer DNA material directly from a recombinant bacterial plasmid into the host cell. DNA transferred by this method is stably integrated into the genome of the recipient plant cells, and plant regeneration in the presence of a selective marker (e.g. antibiotic resistance) produces transgenic plants.

Alternatively, for monocotyledon plants, such as rice (Lin and Assad-Garcia, 1996), corn, and wheat which may not be susceptible to Agrobacterium-mediated bacterial conjugation, DNA may be inserted by such techniques as microinjection, electroporation or chemical transformation of plant cell protoplasts (Paredes-Lopez, 1999 and references therein), or particle bombardment using biolistic devices (Miesfeld, 1999; Paredes-Lopez, 1999; and references therein). Monocotyledon crop plants have now been increasingly transformed with Agrobacterium (Hiei, 1997) as well.

Further, development of the transgenic plant of the present invention may be carried out by the technique of homologous recombination, such as is described, for example, by Zhu et al., (2000).

In order to insert a gene encoding the mutant ALS enzyme of the present invention (i.e. into a host plant, the gene may be incorporated into a suitable construct such as a vector. Such vectors are well known to those of skill in the art, and are used primarily to facilitate handling and manipulation of the gene or gene fragment. Techniques for manipulating DNA sequences (e.g. restriction digests, ligation reactions, and the like) are well known and readily available to those of skill in the art. For example, see Brown, 1998 and Sambrook, 1989. Suitable vectors for use in the methods of the present invention are well known to those of skill in the art. Such vectors include but are not limited to pBC, pGEM, pUC, etc.

Further, such vector constructs may include various useful elements that are necessary or useful for the expression of the gene. Examples of such elements include promoters operably linked to the gene of interest (e.g strong or inducible promoters), enhancer elements, genes for selection such as antibiotic or other herbicide resistance genes (both cross- and multiple-resistance genes), genes which encode factors necessary or useful for effecting the transformation of plants with the gene of interest, terminators, targeting sequences, codes for affinity tags or antibody epitopes, etc. All such variations of the vector of the present invention are intended to be encompassed by the present invention, so long as the vector houses an ALS gene that encodes an ALS enzyme that displays cross-resistance to multiple herbicides.

There are many host plants which could benefit by being transformed by the methods of the present invention to exhibit resistance to herbicides. Such plants include both mono- and dicotyledon species. While the practice of the present invention is applicable to all plant species, it is especially useful for crop plants such as corn, wheat, soybean, cotton, rice, sorghum, canola, and the like. Further, the exact level of expression of the mutant ALS enzyme may vary somewhat from plant to plant, or among species or varieties of plants that are transformed with a mutant ALS gene of the present invention. However, in general, any plant so transformed is intended to be within the scope of the present invention.

By “transgenic plant” we mean any segment or portion of a plant, at least some cells of which contain a mutant ALS gene of the present invention, and express or previously expressed or are capable of expressing (e.g. upon further development) a mutant ALS enzyme of the present invention. Examples include but are not limited to: single plant cells; the stalks, roots, leaves and flowers of a plant; fruit or seeds produced by the plant; vegetative organs such as rhizomes, stolen, bulbs, tubers, and corms; etc. All such portions of, products of, precursors of, etc. a transgenic plant are intended to be encompassed by the present invention. Further, the term “plant” encompasses crop plants such as vegetables, grasses, bushes and trees that produce berries and fruits, ornamental plants (e.g. roses and other flowering plants), and forage crops (including alfalfa, clover, vetches, pasture and hay), grasses, grains, fiber crops, pulp trees, timber, etc.

The invention is further illustrated in the foregoing non-limiting examples.

### EXAMPLES

### Example 1

### Characterization of ALS Resistance

Seeds from a smooth pigweed (Aniaranthus hybridus L.) population (R11-AMACH) were collected from a field in southeastern Pennsylvania where extreme ALS-inhibitor herbicide selection pressure was imposed over a several year period within continuous soybean production. R11-AMACH was selected naturally with ALS-inhibiting herbicides representative of the SU, IMI, and TP herbicide chemistries.

To establish levels and patterns of ALS resistance, R11-AMACH and an ALS susceptible smooth pigweed biotype (S-AMACH) were screened in the greenhouse with various rates of the ALS-inhibiting herbicides, chlorimuron (SU), thifensulfuron (SU), imazethapyr (IMI), pyrithiobac (POB), and cloransulam-methyl (TP). Rates evaluated were based on a log10 scale that included 0, 1/100×, 1/10×, 1×, 10×, and 100×, where 1× corresponds to the normal use rate in the field. R11-AMACH responded differently to the rate increase as compared to S-AMACH. With all herbicides applied, R11-AMACH showed high-levels of resistance based on the response of the S-AMACH. Visual control, height, biomass, and biomass reduction are presented separately for chlorimuron (Table 3), thifensulfuron (Table 4), imazethapyr (Table 5), pyrithiobac (Table 6), and cloransulam (Table 7). Evaluations and measurements were recorded 3 weeks after herbicide treatment (WAT). Visual control was based on a scale of 0-99%, where 0% represents no control and 99% represents complete control. Biomass represents plant dry weights recorded several days after plants were harvested. Biomass reduction was calculated based on the amount of biomass reduced by herbicide treatment compared to the untreated plant biomass.

Results show R11-AMACH resistance levels above 100 times the normal use rate to both SU herbicides, chlorimuron and thifensulfuron, and to the TP herbicide, cloransulam-methyl. Resistance levels to IMI and POB herbicides, imazethapyr and pyrithiobac, respectively, were greater than 10 times the normal use rate. Results indicated R11-AMACH has target-site cross-resistance to four classes of structurally unrelated chemistries of ALS-inhibiting herbicides, namely SU, IMI, POB, and TP.

TABLE 3
R11-AMACH and S-AMACH visual control, height, biomass, and biomass
reduction 3 WAT with various rates of chlorimuron (SU).

| | Visual Control | | Height | | Biomass | | Biomass Reduction | |
|---|---|---|---|---|---|---|---|---|
| | R11-AMACH | S-AMACH | R11-AMACH | S-AMACH | R11-AMACH | S-AMACH | R11-AMACH | S-AMACH |
| RATE | % | | cm | | g | | % | |
| 0 | 0 | 0 | 33.8 | 30.5 | 3.80 | 3.43 | 0.0 | 0.0 |
| 1/100× | 5 | 9 | 23.5 | 27.8 | 3.42 | 2.37 | 10.0 | 30.9 |
| 1/10× | 6 | 20 | 26.5 | 14.3 | 3.66 | 1.65 | 3.7 | 52.0 |
| 1× | 15 | 81 | 21.8 | 4.8 | 3.67 | 0.14 | 3.5 | 95.8 |
| 10× | 18 | 99 | 23.5 | 1.0 | 3.15 | 0.05 | 17.1 | 98.6 |
| 100× | 39 | 99 | 14.8 | 1.3 | 1.64 | 0.11 | 56.8 | 96.7 |

TABLE 4
R11-AMACH and S-AMACH visual control, height, biomass, and biomass
reduction 3 WAT with various rates of thifensulfuron (SU).

| | Visual Control | | Height | | Biomass | | Biomass Reduction | |
|---|---|---|---|---|---|---|---|---|
| | R11-AMACH | S-AMACH | R11-AMACH | S-AMACH | R11-AMACH | S-AMACH | R11-AMACH | S-AMACH |
| RATE | % | | cm | | g | | % | |
| 0 | 0 | 0 | 33.8 | 30.5 | 3.80 | 3.43 | 0.0 | 0.0 |
| 1/100× | 4 | 22 | 36.0 | 25.0 | 4.21 | 1.76 | −10.8 | 48.7 |
| 1/10× | 9 | 47 | 32.5 | 11.0 | 4.45 | 0.85 | −17.1 | 75.2 |
| 1× | 22 | 98 | 23.0 | 2.0 | 3.48 | 0.07 | 8.4 | 98.0 |
| 10× | 38 | 99 | 15.8 | 0.3 | 2.25 | 0.04 | 40.8 | 98.8 |
| 100× | 64 | 99 | 6.8 | 0.5 | 0.58 | 0.03 | 84.7 | 99.1 |

TABLE 5
R11-AMACH and S-AMACH visual control, height, biomass, and biomass
reduction 3 WAT with various rates of imazethapyr (IMI).

| | Visual Control | | Height | | Biomass | | Biomass Reduction | |
|---|---|---|---|---|---|---|---|---|
| | R11-AMACH | S-AMACH | R11-AMACH | S-AMACH | R11-AMACH | S-AMACH | R11-AMACH | S-AMACH |
| RATE | % | | cm | | g | | % | |
| 0 | 0 | 0 | 33.8 | 30.5 | 3.80 | 3.43 | 0.0 | 0.0 |
| 1/100× | 2 | 12 | 32.8 | 25.3 | 4.11 | 1.98 | −8.2 | 42.3 |
| 1/10× | 9 | 58 | 28.0 | 10.3 | 3.00 | 0.63 | 21.1 | 81.6 |
| 1× | 16 | 97 | 20.3 | 2.3 | 2.62 | 0.06 | 31.0 | 98.3 |
| 10× | 62 | 99 | 6.8 | 0.3 | 0.50 | 0.10 | 86.8 | 97.1 |
| 100× | 95 | 95 | 3.3 | 2.3 | 0.15 | 0.06 | 96.1 | 98.3 |

TABLE 6
R11-AMACH and S-AMACH visual control, height, biomass, and biomass
reduction 3 WAT with various rates of pyrithiobac (POB).

| | Visual Control | | Height | | Biomass | | Biomass Reduction | |
|---|---|---|---|---|---|---|---|---|
| | R11-AMACH | S-AMACH | R11-AMACH | S-AMACH | R11-AMACH | S-AMACH | R11-AMACH | S-AMACH |
| RATE | % | | cm | | g | | % | |
| 0 | 0 | 0 | 33.8 | 30.5 | 3.80 | 3.43 | 0.0 | 0.0 |
| 1/100× | 7 | 16 | 26.5 | 21.8 | 2.78 | 1.73 | 26.8 | 49.6 |
| 1/10× | 21 | 59 | 22.0 | 8.8 | 2.42 | 0.37 | 36.3 | 89.2 |
| 1× | 30 | 99 | 17.8 | 2.0 | 2.38 | 0.07 | 37.4 | 98.0 |
| 10× | 48 | 99 | 11.0 | 1.8 | 1.07 | 0.12 | 71.8 | 96.5 |
| 100× | 97 | 99 | 2.5 | 0.5 | 0.07 | 0.04 | 98.2 | 98.8 |

TABLE 7
R11-AMACH and S-AMACH visual control, height, biomass, and biomass
reduction 3 WAT with various rates of cloransulammethyl (TP).

| | Visual Control | | Height | | Biomass | | Biomass Reduction | |
|---|---|---|---|---|---|---|---|---|
| | R11-AMACH | S-AMACH | R11-AMACH | S-AMACH | R11-AMACH | S-AMACH | R11-AMACH | S-AMACH |
| RATE | % | | cm | | g | | % | |
| 0 | 0 | 0 | 46.8 | 34.4 | 10.83 | 8.53 | 0.0 | 0.0 |
| 1/100× | 0 | 97 | 47.8 | 0.7 | 10.78 | 0.02 | 0.5 | 99.8 |
| 1/10× | 23 | 91 | 34.5 | 3.6 | 8.16 | 0.25 | 24.7 | 97.1 |
| 1× | 16 | 96 | 37.6 | 1.3 | 7.49 | 0.08 | 30.8 | 99.1 |
| 10× | 39 | 99 | 32.0 | 0.0 | 6.44 | 0.00 | 40.5 | 100.0 |
| 100× | 63 | 99 | 12.2 | 0.0 | 2.68 | 0.00 | 75.3 | 100.0 |

### Example 2

### Isolation and Sequencing of Herbicide-Resistant ALS Enzymes

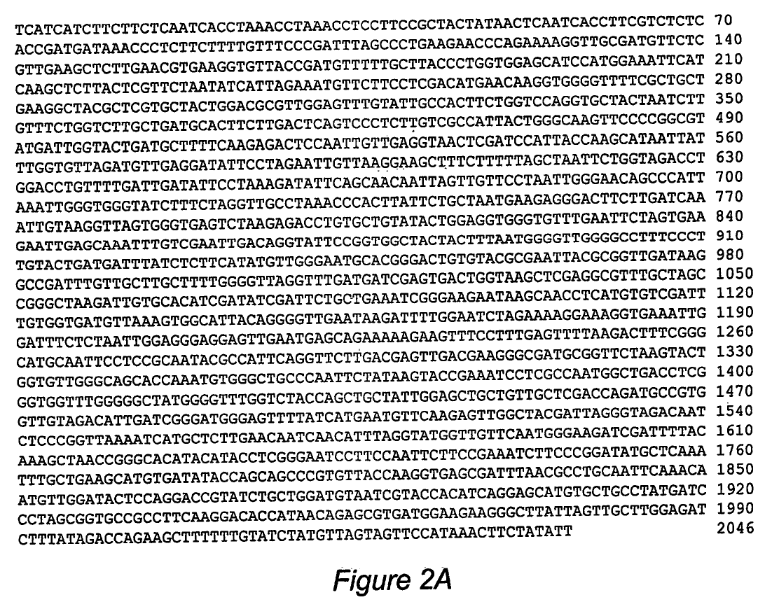

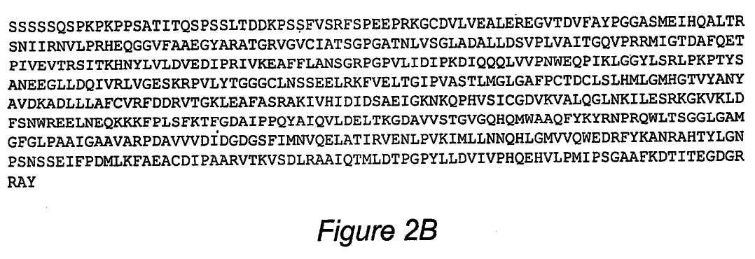

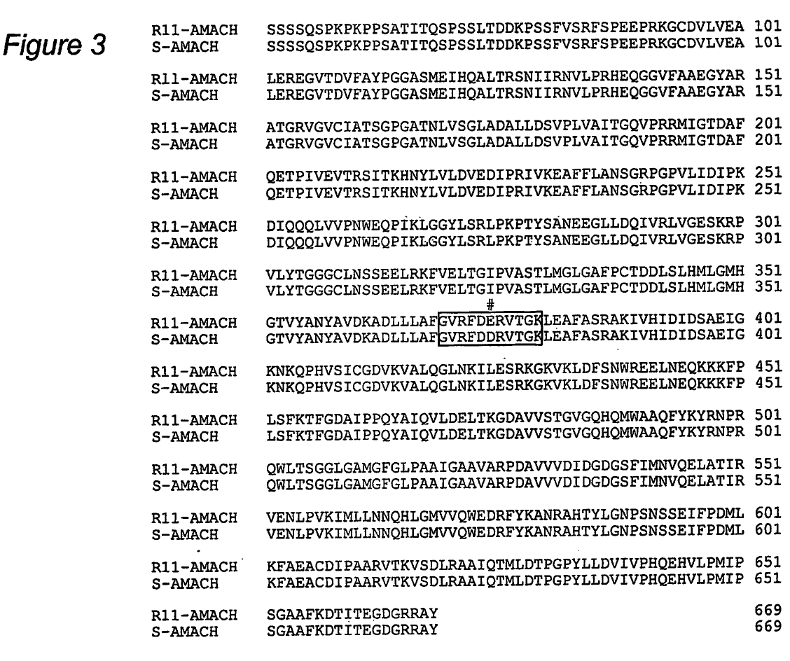

To establish why R11-AMACH exhibited high-levels of resistance to four classes of ALS-inhibiting herbicides, ALS enzymes from R11-AMACH and S-AMACH were isolated and sequenced. The R11-AMACH nucleotide sequence is presented in FIG. 1a (SEQ ID NO: 1) and the corresponding protein in FIG. 1b (SEQ ID NO: 2). The nucleotide sequence of S-AMACH is presented in FIG. 2a (SEQ ID NO: 3) and corresponding protein in FIG. 2b (SEQ ID NO: 4). No nucleotide differences were observed between R11-AMACH and S-AMACH in any of the five previously reported conserved domains known to confer ALS resistance in higher plants. However, a single amino acid difference was discovered in the R11-AMACH biotype ALS that occurred in a conserved region previously unreported to confer ALS resistance in higher plants (FIG. 3, SEQ ID NO: 5). This region consists of the amino acid residues, GVRFDDRVTGK, (SEQ ID NO: 6) which are identical to that of corn (Zea mays), cotton (Gossypium hirsutum), canola (Brassica napus), rice (Oryza sativa), tobacco (Nicotiana tabacum), and Arabidopsis thaliana. The conserved region corresponds to positions 371 to 381 of the Arabidopsis ALS coding sequence. At position 375 of the smooth pigweed ALS amino acid sequence, S-AMACH contained an aspartic acid residue, whereas R11-AMACH contained a glutarnic acid residue (FIG. 3). The amino acid change was a result of a single point mutation in the nucleotide sequence of R11-AMACH where A replaced T in the sequence GAT encoding for aspartic acid (underlined residue is point of mutation).

This invention provides a functional ALS enzyme in higher plants with the amino acid sequence described in FIG. 1b, which confers cross-resistance to ALS-inhibiting herbicides comprising four (or more) structurally unrelated chemistries.

### Example 3

### Enzyme Assay Research

The enzymes from R11-AMACH and S-AMACH were purified and assayed to establish activity and resistance characteristics on the enzyme level. Purification was accomplished by methodology similar to that of Hill et al., (1997). Briefly, large quantities of the enzyme were produced in an expression vector in E. coli in which the recombinant protein was fused to a 6× histidine tag (HIS). Cells were lysed, and the soluble protein fraction purified by differential centrifugation and subsequently passing the protein solution over a nickel column to bind the HIS tag. The ALS protein was eluted from the column, the HIS tag cleaved and the final ALS protein purified from small impurities by passage over a size exclusion column. Activity was assayed using the discontinuous colorimetric assay as described by Singh et al. (1988).

The results showed that resistance levels of R11-AMACH enzyme to SU, IMI, POB, TP and sulfonylamino-carbonyl-triazolinone herbicides were greater than 5 times the concentrations that inhibit the S-AMACH enzyme.

### Example 4

### Development of Transgenic Crop Plants that are Cross-Resistant to Multiple Herbicides

The mutant ALS gene was amplified from genomic DNA utilizing primers designed at the 5′ and 3′ ends to contain both start and stop codons, as well as restriction sites to be used for ligation into a suitable cloning vector. The complete vector contains an appropriate promoter, antibiotic resistance, and the ALS gene. The complete vector was transformed into Agrobacterium tumefaciens. The floral dip method (Clough and Bent (1998) was used for Agrobacterium-mediated transformation into Arabidopsis thaliana. Seed collected from these Arabidopsis were grown on selective media for transgenic plant selection. Furthermore, R11-AMACH plants germinated and grew normally on media containing IMI herbicides at concentrations that completely inhibited growth of wild-type and S-AMACH plants.

Plants surviving the selective media were grown for seed to evaluate resistance characteristics. ALS-inhibiting herbicides were applied at various rates to establish resistance characteristics of the transgenic plants. Transgenic Arabidopsis plants carrying this ALS gene were resistant to all sulfonylurea, imidazolinone, pyrimidinyloxybenzoate, and triazolopyrimidine herbicides.

Transformation and evaluation of crop plants will follow similar methods as those employed with Arabidopsis. Soybean and cotton may be transformed, however, with the use of a particle gun to introduce foreign DNA into the genome rather than using Agrobacterium.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

Bernasconi et al., J. Biol. Chem. (1995) 270:17381-17385.
Boutsalis et al., Pestic. Sci. (1999) 55:507-516.
Brown, T. A., Gene Cloning (1998) Stanley Thomes Ltd. United Kingdon, publisher.
Clough and Bent, Plant J. (1998) 16:735-743.
   Devine and Shukla, Crop Prot. (2000) 19:881-889.
   Devine and Eberlein, Herbicide Activity: Toxicology, Biochemistry and Molecular Biology (1997) 159-185.
   Guttieri et al., Weed Sci. (1992) 40:670-676.
   Guttieri et al., Weed Sci. (1995) 43:175-178.
   Hartnett et al. (1990) A. C. S. Symp. Ser. Am. Chem. Soc. (1990) 421:459-473
Hiei, Y. et al. Plant Mol. Biol. 1997, 35:205-218.
Hill, C. M., Pang, S. S., and Duggleby, R. G. (1997) Biochem. J. 327:891-898.
Lin, J.-J., Assad-Garcia, N. and Kuo, J. Plant Science 1995; 109:171-177.
   Lin, J.-J. and Assad-Garcia, In Vitro 1996; 32:35A-36A.
   Miesfeld, R. L. Applied Molecular Genetics, 1999; Wiley-Liss, publisher, pp. 205-235.
   Paredes-Lopez, ed. Molecular Biotechnology for Plant Food Production, Technomic Publishing, Inc. 1999; 83-86.
   Sambrook, J., E. F. Fritsch, and T. Maniatis, Molecular Cloning: a Laboratory Manual 2^{nd} edition (1989), Cold Spring Harbor Press, New York, publisher.
   Singh et al., Anal. Biochem. (1988) 171: 173-179.
   Westerfield, J. Biol. Chem. (1945) 161:495-502.
Woodworth et al., Plant Physiol. (1996) 111:415
Woodworth et al., Plant Physiol. (1996) 111:S105
Wright et al., Weed Sci. (1998) 46:13-23.
   Zhu et al., Nat. Biothechol. (2000) 18:555-558.

We claim:

1. A substantially purified acetolactate synthase (ALS) enzyme that confers, in a plant, cross-resistance to multiple herbicides.

2. The ALS enzyme of claim 1, wherein the sequence of said ALS enzyme is SEQ ID NO: 1, or a fragment thereof with ALS activity.

3. The ALS enzyme of claim 1, wherein at least two of said multiple herbicides are selected from the group consisting of sulfonylurea, imidazolinone, pyrimidinyloxybenzoate, triazolopyrimidine and sulfonylamino-carbonyl-triazolinone herbicides.

4. A substantially purified ALS gene encoding an ALS enzyme that confers, in a plant, cross-resistance to multiple herbicides.

5. The ALS gene of claim 4, wherein said gene is SEQ ID NO: 2 or a fragment thereof encoding a polypeptide with ALS activity.

6. The ALS gene of claim 4, wherein at least two of said multiple herbicides are selected from the group consisting of sulfonylurea, imidazolinone, pyrimidinyloxybenzoate, triazolopyrimidine and sulfonylamino-carbonyl-triazolinone herbicides.

7. A method of conferring cross-resistance to multiple herbicides to a plant, comprising the step of introducing into said plant an expressible gene encoding an ALS enzyme that exhibits cross-resistance to multiple herbicides, wherein said step of introducing confers cross-resistance to multiple herbicides to said plant.

8. The method of claim 7, wherein said gene is SEQ ID NO: 1, or a fragment thereof that encodes a polypeptide having ALS activity.

9. A transgenic plant that is cross-resistant to multiple herbicides, comprised of a host plant that contains an expressible gene that is not naturally present in said plant, said gene encoding an ALS enzyme that confers cross-resistance to multiple herbicides.

10. The plant of claim 9, wherein said gene is SEQ ID NO:2, or a fragment thereof that encodes a polypeptide having ALS activity.

11. The transgenic plant of claim 9, wherein at least two of said multiple herbicides are selected from the group consisting of sulfonylurea, imidazolinoiie, pyrimidinyloxybenzoate, triazolopyrimidine and sulfonylamino-carbonyl-triazolinone herbicides.

12. The transgenic plant of claim 9 wherein said plant is selected from the group consisting of Arabidopsis, corn, cotton, soybean, rice, wheat, and forage crops.

13. The transgenic plant of claim 9, wherein said ALS enzyme has an aspartic acid to glutamic acid substitution at position six of a conserved sequence GVRFDDRVTGK (SEQ ID NO: 6).
